(19) United States
(12) Patent Application Publication
(10) Pub. No.: US 2007/0219479 A1
(43) Pub. Date: Sep. 20, 2007

(54) Tampon applicator for insertion of a lubricated tampon

(76) Inventor: Hedy E. Tasbas, Fairport, NY (US)

Correspondence Address: HOWARD J. GREENWALD P.C., 349 W. COMMERCIAL STREET SUITE 3075, EAST ROCHESTER, NY, 14445-2408, US

(21) Appl. No.: 11/384,788

(22) Filed: Mar. 20, 2006

(51) Int. Cl. A61F 13/20

(52) U.S. Cl. 604/12; 604/15

(57) ABSTRACT

Disclosed in this specification is a tampon insertion assembly comprised of a flexible tampon applicator with a hollow cavity and lubricant disposed therein. The applicator is flexible such that a human hand can depress a portion of the applicator and thus cause an absorbent material contained within to move within the cavity and be coated by a lubricant.

### FIELD OF THE INVENTION

This invention pertains to a means for collecting bodily fluids. More specifically, this invention relates to an absorbent pad for use as a catamenial device with lubricating means for facilitating insertion and removal of such an absorbent pad.

### BACKGROUND OF THE INVENTION

The insertion and removal of tampons is uncomfortable process for many women. The insertion of a tampon often requires the use of untidy lubricants and is otherwise awkward for the user. Similarly, during use, the tampon often becomes frictionally engaged to the vaginal walls. During removal of the tampon, such an engagement makes removal of the tampon painful for many women. Numerous attempts to solve these problems have been made in the prior art, but none has proven entirely satisfactory.

International application WO 02/017844 to Kreutz (Feminine Hygiene Kit) discloses a kit comprised of feminine hygiene products solid in combination with lubricants and other accessories. Similarly, U.S. Pat. No. 4,077,409 to Murray (Encapsulated Catamenial Device) teaches a feminine hygiene product which is adapted to provide lubrication to the tip of the tampon. These and similar devices are deficient in that they only provide lubrication to the distal (top) end of the tampon. Such a configuration promotes easy insertion, but does nothing to alleviate the pain of withdrawing such a device. The content of both of the aforementioned documents is hereby incorporated by reference into this specification.

U.S. Pat. No. 6,746,418 to Pauley (Methods of Lubricating a Tampon and a Tampon Lubricated Thereby) illustrates the use of wrapped tampons, wherein lubricant is disposed within the wrapping. This device is deficient in that it lubricates only the tampon applicator, and not the absorbent material within the applicator. Such a configuration promotes insertion, but does nothing to alleviate the pain of withdrawing such a device. The content of the aforementioned patent is hereby incorporated by reference into this specification.

U.S. Pat. No. 4,421,504 to Kline (Lubricating Object Injector Utilizing a single Plunger) discloses a device for injecting suppositories into a body cavity; primarily the anal cavity. Upon activation of the plunger, the surface of the applicator becomes lubricated, thus promoting easy insertion through a sphincter. Like the previously mentioned prior art, this device does not permit complete lubrication of the object to be delivered. The content of the aforementioned patent is hereby incorporated by reference into this specification.

Other prior art considered of some importance to this application includes U.S. Pat. No. 2,739,593 to McLaughlin (Medicated Vaginal Tampons); U.S. Pat. No. 4,026,292 to Hutchins (Tampon having a Mensesphilic Foam Treated with a Liquid Lubricant); U.S. Pat. No. 3,756,238 to Hanke (Self-lubricating Compound for use in Hygienic and Medical Applications, and Coated Tampons and Suppositories Made Therefrom); U.S. Pat. No. 3,717,149 to Morane (Injector Package for Catamenial Tampon); U.S. Pat. No. 3,674,025 to Bleuer (Tampon Assembly); U.S. Pat. No. 4,056,103 to Kaczmarzyk (Wrapper Structure for Tampons Containing Superabsorbent Material); U.S. Pat. No. 4,061,145 to DesMarais (Absorbent Foam Articles and Method of Manufacture); U.S. Pat. No. 3,994,298 to Des Marais (Foam Aggregate Catamenial Tampon); RE30,029 to Smith (Absorbent Mass of Alloy Fibers of Regenerated Cellulose and Polyacrylic Acid Salt of Alkali-Metals of Ammonium); U.S. Pat. No. 3,844,287 to Smith (Absorbent Mass of Alloy Fibers of Regenerated Cellulose and Polyacrylic Acid Salt of Alkali-Metals or Ammonium); U.S. Pat. No. 3,995,636 to Murray (Constrained Catamenial Device Employing Adhesive Coating); U.S. Pat. No. 6,939,289 to Zunker (Elliptical Applicator System); U.S. Pat. No. 6,610,025 to Berg (Tampon Applicator Arrangement); U.S. Pat. No. 6,592,540 to DeCarlo (Tampon Applicator with Lubricated Tip); U.S. Pat. No. 6,524,269 to McNamara (Lubricated Tampon); U.S. Pat. No. 4,690,671 to Coleman (Lubricated Tampon Inserter); U.S. Pat. No. 3,791,385 to Davis (Catamenial Device and Applicator Thereof); U.S. Pat. No. 3,335,726 to Maranto (Lubricating Tampon); U.S. Pat. No. 5,533,990 to Yeo (Tampon Exhibiting Low Frictional Drag); U.S. Pat. No. 3,139,886 to Tallman (Tampon Applicator with Lubricating Feature); U.S. Pat. No. 4,428,747 to Friese (Tampon Blister Pack); U.S. Pat. No. 4,312,348 to Friese (Tampon Pack with a Lubricant Depot in the Packaging); United States patent applications 2002/0026140A1 to McNamara (Lubricated Tampon); 2002/0128590A1 to Decarlo (Tampon Applicator with Lubricated Tip); and 2005/0186427A1 to Gatto (Lubricious Coated Applicator). The content of each of the aforementioned patents and patent applications is hereby incorporated by reference into this specification.

It is an object of this invention to provide at least one of the following: a tampon applicator that allows the user to coat the entire surface of the absorbent material such that the material is more easily removed from a vagina; a tampon insertion assembly that lubricates not just the applicator, but also the absorbent material; a tampon insertion assembly that lubricates not just the tip of the absorbent material, but lubricates the entire outer surface of the material.

### SUMMARY OF THE INVENTION

In accordance with the present invention, there is provided a tampon insertion assembly comprised of a flexible tampon applicator with a hollow cavity, an absorbent material, and lubricant disposed therein. In one embodiment, the lubricant is placed into the hollow cavity by the user. The applicator is flexible such that a human hand can depress a portion of the applicator and thus cause the absorbent material contained within to move within the cavity and be coated by a lubricant.

### BRIEF DESCRIPTION OF THE DRAWINGS

The invention will be described by reference to the following drawings, in which like numerals refer to like elements, and in which:

FIG. 1 is a schematic diagram of one tampon assembly of the present invention;

FIG. 2 is another schematic diagram of one such tampon assembly;

FIG. 3A, FIG. 3B, and FIG. 3C are depictions of one method for the lubrication of an absorbent material using the present invention;

FIG. 4A, FIG. 4B, and FIG. 4C are illustrations of one methods of the insertion of the lubricated, absorbent material of this invention;

FIG. 5 is a flow diagram depicting one process for use with the present invention; and

FIG. 6A and FIG. 6B are schematic diagrams of two kits for use with the present invention.

The present invention will be described in connection with a preferred embodiment, however, it will be understood that there is no intent to limit the invention to the embodiment described. On the contrary, the intent is to cover all alternatives, modifications, and equivalents as may be included within the spirit and scope of the invention as defined by the appended claims.

### DESCRIPTION OF THE PREFERRED EMBODIMENTS

For a general understanding of the present invention, reference is made to the drawings. In the drawings, like reference numerals have been used throughout to designate identical elements.

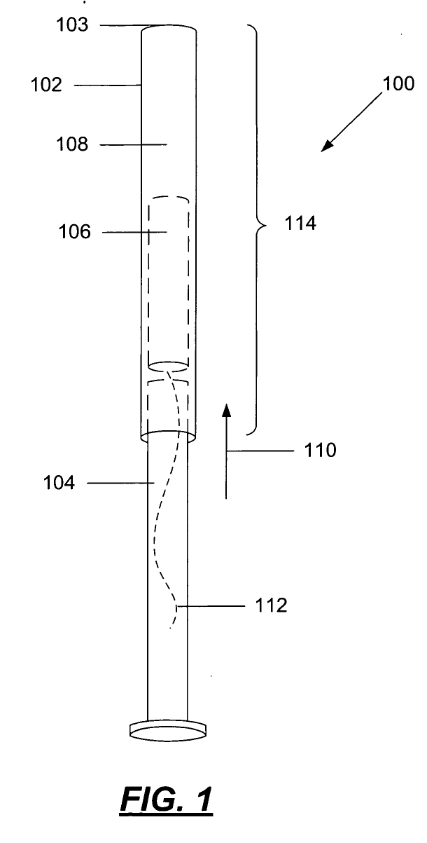

FIG. 1 is a depiction of tampon insertion assembly 100. Assembly 100 is comprised of applicator 102 and plunger 104. Applicator 102 has a hollow cavity 114. Such applicators are known in the art. Reference may be had to U.S. Pat. No. 4,690,671 to Coleman (Lubricated Tampon Inserter) and the like. Hollow cavity 114, in turn, contains an absorbent material 106 (which is connected to retrieval string 112) and a receiving space 108 configured to receive a lubricant (not shown). Applicator 102 has an opening 103 at its distal end and is configured to receive plunger 104 at its proximal end. Operation of plunger 104 in the direction of arrow 110 causes absorbent material 106 to pass through receiving space 108 and out opening 103.

Applicator 102 is flexible. In one embodiment, applicator 102 is flexible such that depression of the walls of the applicator at opposing points causes the two walls to be contiguous without breaking the applicator.

In one embodiment, applicator 102 is also transparent such that the contents of the applicator 102 can be viewed through the walls of the applicator. In one such embodiment, applicator 102 is constructed from a flexible, transparent plastic known to those skilled in the art. For example, in one embodiment, applicator 102 is constructed from low-density polyethylene. Such polyethylene has found widespread use in disposal pipettes. Other suitable flexible, transparent plastics may also be used so long as such plastics flex upon application of pressure from a human hand.

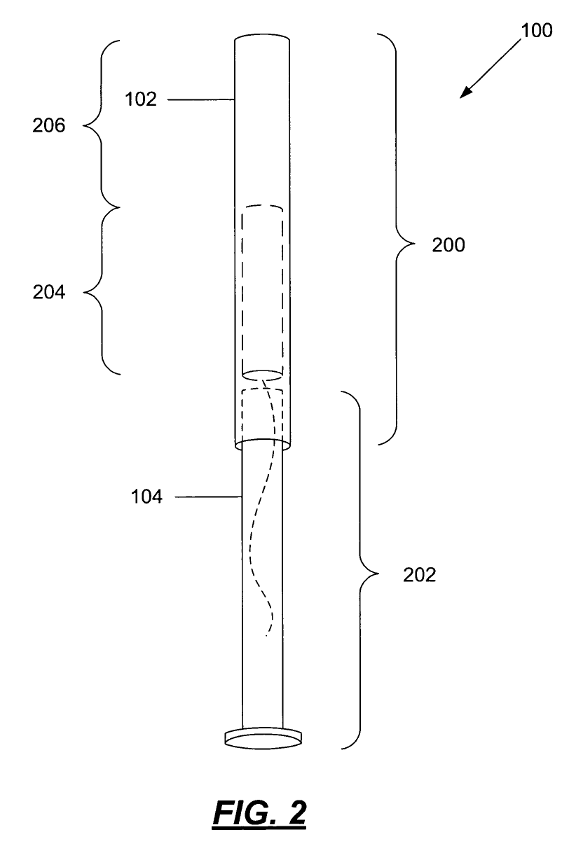

FIG. 2 is an illustration of assembly 100 wherein the dimensions of the components are illustrated. In the embodiment depicted in FIG. 2, applicator 102 is comprised of a hollow cavity length 200 which is comprised of a receiving space length 206 and an absorbent material length 204. In the embodiment depicted, receiving space length 206 is substantially the same as absorbent material length 204. Such a configuration promotes the lubrication of absorbent material 106 (see FIG. 1) by providing sufficient space to move the absorbent material 106. In one embodiment, receiving space length 206 is from about 7 centimeters to about 13 centimeters, and thus absorbent material length 204 is about the same length. In such an embodiment, hollow cavity length 200 is at least about 14 centimeters. In another embodiment, receiving space length 206 is from about 9 centimeters to about 11 centimeters. As can be seen in FIG. 2, the hollow cavity length 200 may be longer than the sum of the receiving space length 206 and the absorbent material length 204. A portion of the length of hollow cavity length 200 may be used to receive plunger 104.

Referring again to FIG. 2, plunger 104 is comprised of a plunger length 202. In the embodiment depicted, plunger length 202 is substantially the same as hollow cavity length 200. Such a configuration ensures that the absorbent material 106 will be expelled from hollow cavity 114 (see FIG. 1) upon operation of plunger 104. In one embodiment, plunger length 202 is at least about 80 percent of the length of hollow cavity length 200. In another embodiment, plunger length 202 is at least about 90 percent of the length of hollow cavity length 200.

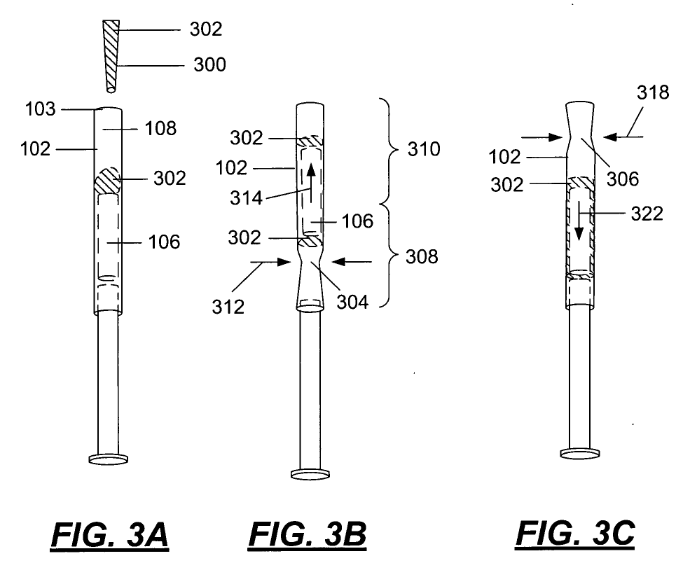

FIGS. 3A, 3B and 3C are illustrations which show one means for applying lubrication using the present invention. Generally, the user places lubricant into a receiving area of the tampon applicator. The applicator is flexible such that squeezing the applicator walls causes the absorbent material within the applicator to pass through the lubricant. By repeatedly and intermittently flexing the applicator at various points, the absorbent material is repeatedly exposed to the lubricant, and thus the entire surface of the absorbent material is coated. It is preferred that the width of applicator 102 be such that the lubricant 302 be able to around absorbent material 106. In one embodiment, the width of applicator 102 is within a range of from about 1 centimeter to about 3 centimeters. In another embodiment, the width of applicator 102 is about 2 centimeters. In another embodiment, the lubricant 302 passes around absorbent material 106 through conduits, such as those conduits disclosed in U.S. Pat. No. 4,690,671.

As depicted in FIG. 3A, lubricant 302 shown in FIG. 3A is placed in receiving area 108 by the user. In another embodiment, not shown, the manufacturer of the assembly disposes lubricant 302 in receiving area 108, thus the lubricant is already in the receiving area 108 when the end user opens the product. In either embodiment, pressure is applied to a proximal point 304 (see FIG. 3B) which forces absorbent material 106 into intimate contact with the lubricant 302. In the embodiment depicted, the two walls of proximal point 304 are not contiguous. It would be apparent to one skilled in the art that applicator of a greater amount of force will cause the walls to become contiguous. Application of pressure to a distal point 306 (see FIG. 3C) reverses the direction of absorbent material 106 and promotes lubrication of the absorbent material 106. The steps of applying pressure to proximal point 304 and distal point 306 (see FIG. 3C) may be repeated until the user is satisfied with the degree of lubrication. It is advantageous that the applicator 102 be comprised of a transparent, flexible plastic. The flexible nature of the plastic permits the manipulation of absorbent material 106 while the transparency ensures the user can see that the lubricant 302 is properly coating the material 106.

As shown in FIG. 3A, the first step of the lubrication process is the addition of lubricant 302 to receiving space 108. In the embodiment depicted, lubricant 302 is added through opening 103 of applicator 102 by lubricant applicator 300 such that the distal end of absorbent material 106 is coated. Lubricant applicator 300 may be, for example, a squeezable bottle of lubricant 302. In another embodiment, not shown, the lubricant is also placed on the external surface of applicator 102, thus facilitating the insertion of the applicator into a vagina. Suitable lubricants are known to those skilled in the art. For example one may use an aqueous glycerin solution. One such solution is the commercially available K-Y™ Jelly solid by the Johnson & Johnson Corporation. Other suitable lubricants would be apparent to those skilled in the art and are considered within the scope of this invention. As used in this specification, the term “lubricant” includes liquid, semi-liquid, and gel like fluids that decrease the coefficient of friction of a surface. It is desirable that the lubricant be biocompatible, sterile, hygienic and adapted for use in a vaginal orifice. Other suitable lubricants that are to be considered within the scope of this invention include those lubricates disclosed in U.S. Pat. No. 4,981,686 to Hardy (Personal Lubricant); U.S. Pat. No. 5,885,591 to Ahmad (Personal Lubricant Compositions); U.S. Pat. No. 6,139,848 to Ahmad (Personal Lubricant Compositions); U.S. Pat. No. 6,144,398 to Ratcliff (Personal lubricant to prevent and stop itching); U.S. Pat. No. 5,902,593 to Kent (Topically Applied Personal Lubricant Containing Benzalkonium Chloride as the Active Ingredient); and the like. The properties of such lubricants that are considered desirable include anti-viral properties (see U.S. Pat. No. 5,208,031 to Kelly entitled Sexual Lubricants Containing Zinc as an Anti-viral Agent); anti-bacterial properties (see U.S. Pat. No. 5,536,743 to Borgman entitled Intravaginal Treatment of Vaginal Infections with Buffered Metronidazole Compositions); anti-fungal or anti-yeast properties (see U.S. Pat. No. 6,277,363 to Ratcliff entitled Method and composition for treating Chlamydia infection); as well as fragrant properties. The content of each of the aforementioned patents is hereby incorporated by reference into this specification.

Referring now to FIG. 3B, once the lubricant 302 has coated the top of absorbent material 105, pressure is applied to a proximal point 304 of applicator 102. Application of pressure to applicator 102 in the direction of arrows 312 causes the walls of the applicator 102 to collapse at such point 304. Such a compression forces absorbent material 106 to travel in the direction of arrow 314 from proximal portion 308 into distal portion 310. As lubricant 302 is disposed within distal portion 310 (i.e. receiving space 108 is within distal portion 310), such traveling coats absorbent material 106 with lubricant 302, thus producing a lubricated, absorbent material. In one embodiment, not shown, the user may also pinch opening 103 shut, thus ensuring absorbent material 106 does not exit opening 103 during the lubrication process. In another embodiment, the amount of pressure applied to proximal point 304 is such that the absorbent material 106 does not exit opening 103, despite opening 103 being un-pinched.

Referring now to FIG. 3C, once the absorbent material 106 is disposed within distal portion 310 (see FIG. 3B), pressure is released from proximal point 304 (see FIG. 3B) and pressure is thereafter applied to distal point 306 in the direction of arrows 318. Such compression forces absorbent material 106 to travel in the direction of arrow 320 from distal portion 310 into proximal portion 308 (see FIG. 3B). By repeatedly and intermittently applying pressure to distal point 304 (see FIG. 3B) and proximal point 306 (see FIG. 3C) the absorbent material 106 may have its entire surface coated with lubricant 302.

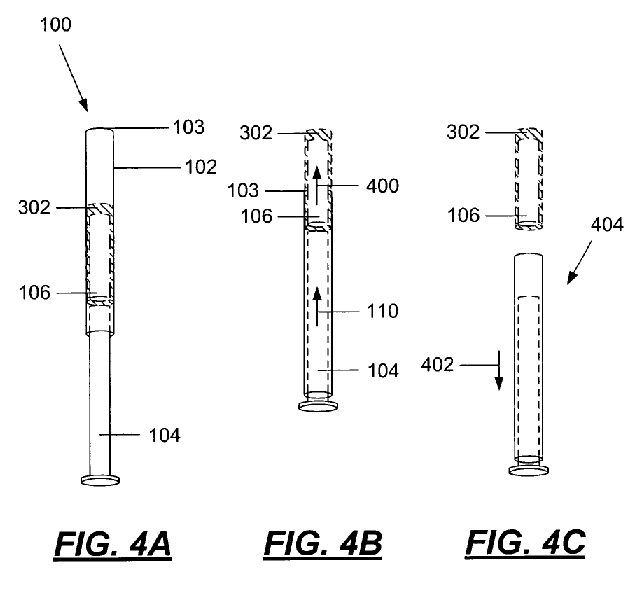

FIGS. 4A, 4B and 4C are depictions of the expulsion of a lubricated, absorbent material from the assembly 100 (see FIG. 1). As shown in FIG. 4A, in the first step of the application process the assembly 100 is disposed within a vaginal cavity such that applicator 102 is contiguous with the vaginal walls. Thereafter the absorbent material 106 is expelled from opening 103.

FIG. 4B depicts another step of the application process, wherein the plunger 104 is depressed in the direction of arrow 110 such that absorbent material 106 travels in the direction of arrow 400, through applicator 102, and is thereafter expelled through opening 103. The external surface of lubricated, absorbent material 106 is at least partially contiguous with the aforementioned vaginal walls. In this manner the lubricated, absorbent material 106 is frictionally engaged with the vaginal walls.

FIG. 4C illustrates the removal of spent applicator 404. The spent applicator 404 is simply removed from the vaginal cavity (not shown) by moving spent applicator 404 in the direction of arrow 402. In one embodiment, the steps illustrated in FIG. 4B and FIG. 4C are separate and distinct steps which occur in different moments in time. In another embodiment, the steps are simultaneous. For example, the depression of the plunger 104 in the direction of arrow 110 occurs at the same moment in time as the spent applicator 404 is moved in the direction of arrow 402. In either event, an absorbent material 106, completely coated with lubricant 302, is delivered into the vaginal cavity (not shown).

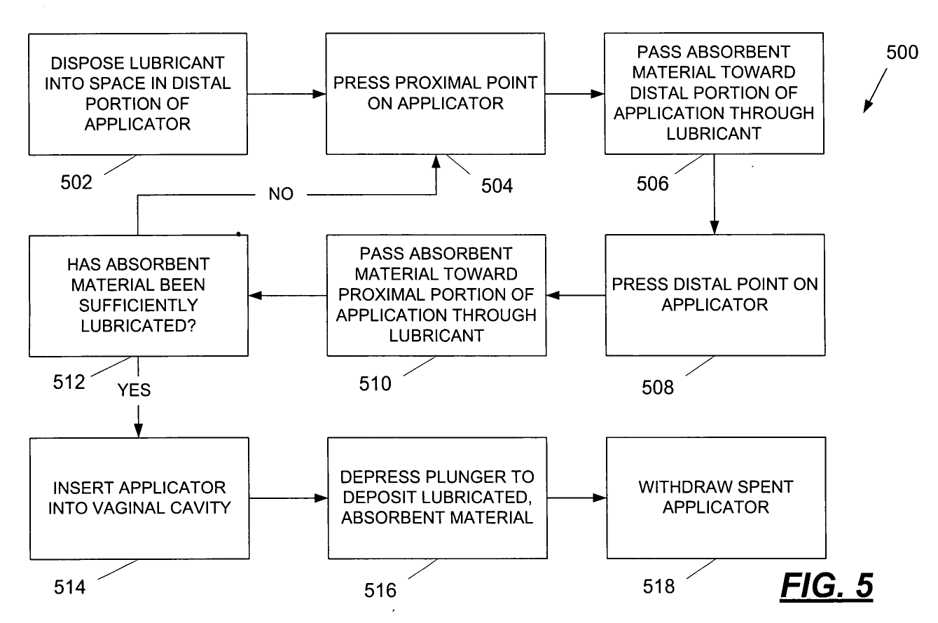

FIG. 5 is a flow diagram of one process 500 for use with the present invention. In the first step 502 of process 500, a lubricant is disposed in a space in the distal portion of a tampon applicator. Reference may be had to FIG. 3A, wherein lubricant 302 is disposed in receiving space 108 of applicator 102. In the second step 504, a proximal point is pressed on the proximal end of the applicator. Such a step is illustrated in FIG. 3B, wherein proximal point 304 is depressed. In step 506, the absorbent material is passed from the proximal portion of the applicator into the distal portion, wherein the lubricant is disposed. Reference may be had to FIG. 3B. Thereafter, the pressure to the proximal point is released (step not shown). In step 508 of the process, which is optional, pressure is applied to a distal point on the applicator. Reference may be had to FIG. 3C, wherein pressure is applied to distal point 306. Such a pressure causes step 510 to be executed, wherein the absorbent material is passed from the distal portion and into the proximal portion of the applicator. Reference may be had to FIG. 3C. In step 512 of process 500, the user inspects the absorbent material to determine if it is sufficiently lubricated. If the lubrication is not sufficient, the user may continue the lubrication process by repeating steps 504 to 510. In another embodiment, not shown, the user adds additional lubricant by repeating steps 502 to 510. If the lubrication is sufficient, the user proceeds to step 514. In step 514 of process 500, the applicator is inserted into a vaginal cavity. In one embodiment, the top of the applicator is curved, thus promoting easy insertion into the cavity. In step 516 of the process, the plunger is depressed, and the absorbent material is deposited into the vagina. In step 518 the spent applicator is removed. In one embodiment, step 516 and step 518 occur simultaneously.

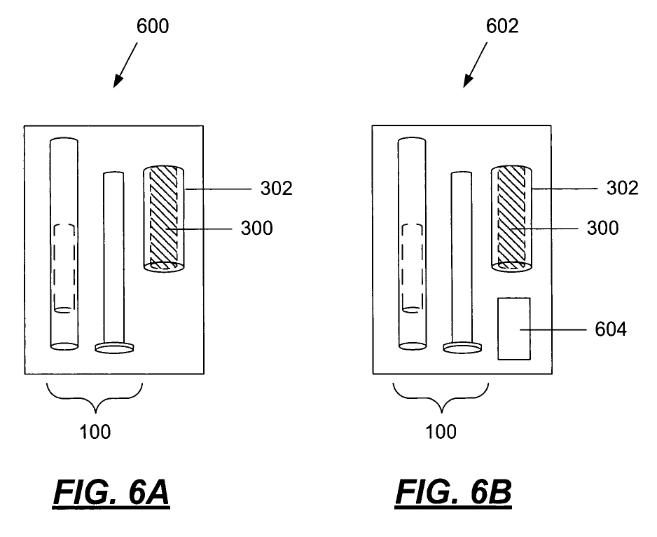

FIG. 6A and FIG. 6B are depictions of two kits for use with the present invention. FIG. 6A is an illustration of kit 600 that is comprised of tampon insertion assembly 100. This kit also includes lubricant 302 disposed inside of lubricant applicator 300. In the embodiment depicted, the plunger is not connected to the applicator. Such a disconnected configuration reduces the packing space required. The user is able to easy reassemble the assembly 100 prior to use. The kit may also be comprised of various accessory items.

FIG. 6B is an illustration of kit 602 that is substantially identical to kit 600 expect in that accessory 604 is included with the kit. Accessory 604 may be, for example, a sanitary napkin, mirror, finger glove, glove, instruction manual or other accessory typically found with similar prior art kits. Reference may be had to International Publication Number WO 02/017844 and the like.

In another embodiment, not shown, the kit is comprised of a plurality of tampon insertion assemblies 100 and a large bottle of lubricant applicator 300. Such a large bottle contains sufficient lubricant 302 to lubricate the plurality of assemblies 100, thus permitting multiple uses from a single kit.

It is therefore, apparent that there has been provided, in accordance with the present invention, a method and apparatus for the insertion of a lubricated tampon. While this invention has been described in conjunction with preferred embodiments thereof, it is evident that many alternatives, modifications, and variations will be apparent to those skilled in the art. Accordingly, it is intended to embrace all such alternatives, modifications and variations that fall within the spirit and broad scope of the appended claims.

1. A tampon insertion assembly comprising:
a. an absorbent material with an absorbent material length;
b. a flexible tampon applicator with an applicator length comprising a hollow cavity, wherein said hollow cavity has a proximal portion and a distal portion, wherein;
i. said distal portion is comprised of an opening and a receiving space with a receiving space length configured to receive a lubricant;
ii. said absorbent material is disposed within said proximal portion;
iii. said receiving space length is about equal to said absorbent material length;
c. a plunger, operatively configured to be disposed in said proximal portion of said hollow cavity or said applicator such that operation of said plunger causes said lubricated, absorbent material to move from said proximal portion, through said distal portion, and thereafter out said opening.

2. The tampon insertion assembly as recited in claim 1, wherein said tampon applicator is flexible.

3. The tampon insertion assembly as recited in claim 2, wherein said receiving space length is from about 7 centimeters to about 13 centimeters.

4. The tampon insertion assembly as recited in claim 3, wherein said flexible plastic is transparent.

5. (canceled)

6. (canceled)

7. The tampon insertion assembly as recited in claim 4, further comprising a lubricant wherein said lubricant is comprised of an aqueous lubricant.

8. The tampon insertion assembly as recited in claim 7, wherein said aqueous lubricant is an aqueous glycerin lubricant.

9. The tampon insertion assembly as recited in claim 4, wherein said hollow cavity has a hollow cavity length consisting of said receiving space length and said absorbent material length, and wherein said plunger has a plunger length that is at least about 80 percent of the length of said hollow cavity length.

10. The tampon insertion assembly as recited in claim 9, wherein said flexible plastic is low-density polyethylene.

11. (canceled)

12. (canceled)

13. (canceled)

14. (canceled)

15. (canceled)

16. The tampon application kit comprising;
a tampon insertion assembly comprising,
i. an absorbent material with an absorbent material length;
ii. a flexible tampon applicator with an applicator length comprising a hollow cavity, wherein said hollow cavity has a proximal portion and a distal portion, wherein;
1. said distal portion is comprised of an opening and a receiving space with a receiving space length configured to receive a lubricant;
2. said absorbent material is disposed within said proximal portion;
3. said receiving space length is about equal to said absorbent material length;
4. said tampon applicator is flexible;
iii. a plunger, operatively configured to be disposed in said proximal portion of said hollow cavity of said applicator such that operation of said plunger causes said lubricated, absorbent material to move from said proximal portion, through said distal portion, and thereafter out said opening; and
b. an accessory item.

17. The tampon application kit as recited in claim 16, wherein said flexible plastic is transparent.

18. The tampon application kit as recited in claim 17, wherein said accessory item is a lubricant applicator.

19. The tampon application kit as recited in claim 18, wherein said accessory item is a sanitary napkin.

20. The tampon application kit as recited in claim 19, further comprising a plurality of said tampon insertion assemblies.
